(19) United States
(12) Patent Application Publication
(10) Pub. No.: US 2012/0264633 A1
(43) Pub. Date: Oct. 18, 2012

(54) METHODS FOR DETECTING THROMBOCYTOSIS USING BIOMARKERS

(76) Inventors: Wadie F. Bahou, Setauket, NY (US); Dmitri V. Gnatenko, Coram, NY (US)

(21) Appl. No.: 13/496,567

(22) PCT Filed: Sep. 20, 2010
(86) PCT No.: PCT/US10/49507

§ 371 Date: Jun. 28, 2012

Related U.S. Application Data
(60) Provisional application No. 61/243,808, filed on Sep. 18, 2009.

(51) Int. Cl. C40B 30/04; G01N 21/64; C12Q 1/68

(52) U.S. Cl. 506/9; 435/6.12

(57) ABSTRACT

The present invention relates to a method to determine the gene expression profile of thrombocytotic sensitive genes. The method comprises the following steps; obtaining hematologic samples from subjects in a training set, analyzing the obtained hematologic samples with a microarray, measuring the expression values of each gene on the microarray, performing analysis to identify differentially expressed genes in the training set among three cohorts of thrombocytosis, obtaining hematologic samples from subjects in an independent testing set, and validating the identity of the differentially expressed genes in the independent testing set among the three cohorts of thrombocytosis. The invention also relates to a method to distinguish thrombocytosis cohorts. The method includes the following steps; obtaining a hematologic sample from a subject, determining gene expression of a biomarker subset, analyzing gene expression of the biomarker subset, and classifying the subject into one of three cohorts.

### STATEMENT REGARDING FEDERALLY SPONSORED RESEARCH OR DEVELOPMENT

The work described in this invention was supported in part from grants from the-U.S. National Institutes of Health, grants HL086376, HL49141, HL53665 and HL 76457, the Department of Defense, grant MPO48005 and a NIH Center grant MO1 10710-5. The government may have rights in this invention.

### FIELD OF THE INVENTION

The present invention relates generally to the detection of thrombocytosis in a human, and more particularly, a method utilizing an algorithm that determines phenotypic class using distinct genetic biomarker subsets.

### BACKGROUND OF THE INVENTION

A platelet count above the physiological reference range is considered thrombocytosis. Hematologic criteria for distinguishing among the various causes of thrombocytosis are limited in their capacity to delineate clonal (including essential thrombocythemia “ET”) from non-clonal (including reactive thrombocytosis “RT”) cohorts.

ET is characterized by increased proliferation of megakaryocytes, elevated numbers of circulating platelets, and considerable thrombohemorrhagic events, not infrequently neurological. ET is a myeloproliferative disorder (MPD) subtype microscopically indistinguishable from the larger subset of non-clonal, thrombocytotic disorders associated with a wide array of human diseases. RT is a common condition in medicine, and can be due to a number of serious underlying conditions such as malignancy (cancer), chronic infections, or chronic inflammatory conditions (autoimmune diseases, rheumatoid arthritis, lupus erythematosis, etc.) RT is not an MPD, and usually subsides when the condition is resolved.

The differentiation of ET from RT has important diagnostic and therapeutic implications since thrombohemorrhagic complications arising in non-clonal cohorts such as RT are unusual, in contrast to frequent events in patients with clonal disorders, such as ET. Differentiation is important because patients with ET are predisposed to bleeding or blood clotting abnormalities including stroke and deep vein thrombosis.

Although a cohort for thrombocytosis is evident in many patients, its association with malignancies coupled with the fact that ET remains a diagnosis of exclusion support the need for well-defined diagnostic criteria. No functional or diagnostic test is currently available for ET.

A model using a biomarker gene set expression profile for assigning class in patients with thrombocytosis is desired so that a patient can be accurately classified into a thrombocytotic cohort.

### SUMMARY OF THE INVENTION

In one aspect, the invention is directed to a method to determine the gene expression profile of thrombocytotic sensitive genes. The method comprises the following steps; obtaining hematologic samples from subjects in a training set, analyzing the obtained hematologic samples with a microarray, measuring the expression values of each gene on the microarray, performing analysis to identify a biomarker subset of differentially expressed genes in the training set among three cohorts, obtaining hematologic samples from subjects in an independent testing set, and validating the identity of the differentially expressed genes in the independent testing set among the three cohorts.

In another aspect, the invention is directed to a method to distinguish thrombocytosis cohorts. The method includes the following steps; obtaining a hematologic sample from a subject, determining gene expression of a biomarker subset, analyzing gene expression of the biomarker subset, and classifying the subject into one of three cohorts.

A method to determine the gene expression profile of thrombocytotic sensitive genes is also contemplated. This method comprises; obtaining hematologic samples from subjects in a training set, analyzing the obtained hematologic samples with a microarray, measuring the expression values of each gene on the microarray, performing analysis to identify differentially expressed genes in the training set among three cohorts, obtaining hematologic samples from subjects in an independent testing set, and validating the identity of the differentially expressed genes in the independent testing set among the three cohorts of thrombocytosis.

The method to determine the gene expression profile of thrombocytotic sensitive genes can also include identification of differentially expressed genes. The method to determine the gene expression profile of thrombocytotic sensitive genes can also include identification of up to 15 differentially expressed genes. The method to determine the gene expression profile of thrombocytotic sensitive genes can include identification of the gene expression of a 4 biomarker subset, an 11 biomarker subset, a 15 biomarker subset or variations of the 15 biomarker subset including at least 4 genes. The entire 15 biomarker subset includes the following genes; WASF3, CTNS, HIST1H2AG, ACOT7, LATPM4B, TGFB2, TPM1, H3F3A, APP, NGFRAP1, CLEC1B, HIST1H1A, SRP72, C20orf103 and CRYM.

The method to determine the gene expression profile of thrombocytotic sensitive genes can also include measuring the expression values is by measuring fluorescence intensities. The method to determine the gene expression profile of thrombocytotic sensitive genes can also include identifying differentially expressed genes with use of a combination of the Kruskal-Wallis, non-parametric one-way analysis of variance, nonparametric Wilcoxon rank-sum test and Non-Parametric Linear Discriminant Analysis with a leave-one-out cross-validation analysis. The method to determine the gene expression profile of thrombocytotic sensitive genes can also include identifying differentially expressed genes with use of Non-Parametric Linear Discriminant Analysis with a leave-one-out cross-validation analysis.

Another method is contemplated to distinguish thrombocytosis cohorts, the method including the following steps; obtaining a hematologic sample from a subject, determining gene expression of a biomarker subset, analyzing gene expression of the biomarker subset, and classifying the subject into a cohort. The method to distinguish thrombocytosis cohorts can also include identification of gene expression of a biomarker subset. The biomarker subset can also include identification of the gene expression of a 4 biomarker subset, an 11 biomarker subset, a 15 biomarker subset or variations of the 15 biomarker subset including at least 4 genes. The entire 15 biomarker subset includes the following genes; WASF3, CTNS, HIST1H2AG, ACOT7, LATPM4B, TGFB2, TPM1, H3F3A, APP, NGFRAP1, CLEC1B, HIST1H1A, SRP72, C20orf103 and CRYM.

The method to distinguish thrombocytosis cohorts can also include distinguishing between cohorts selected from the group consisting of: normal subjects, subjects with Essential Thrombocythemia (ET) and subjects with Reactive Thrombocytosis (RT).

The method to distinguish thrombocytosis cohorts can also include determination of gene expression through use of a microarray. The method to distinguish thrombocytosis cohorts can also include determination of gene expression through use of a polymerase chain reaction (PCR). The method to distinguish thrombocytosis cohorts can also include determination of gene expression through use of a PCR wherein that PCR is quantitative real-time reverse-transcription polymerase chain reaction (qRT-PCR).

The method to distinguish thrombocytosis cohorts can also include a hematologic sample of whole blood. The method to distinguish thrombocytosis cohorts can also include a hematologic sample of platelets. The method to distinguish thrombocytosis cohorts can also include determination of gene expression of a subject, wherein that subject is human.

The method to distinguish thrombocytosis cohorts can also include classifying the subject into a cohort with the highest posterior possibility.

The invention advantageously provides a method to identify and diagnose a subject into one of two thrombocytotic cohorts and one normal cohort in an accurate and efficient manner.

### BRIEF DESCRIPTION OF THE FIGURES

FIG. 1. Is a table of the cohorts of several subjects, including several individual values measured for each subject.

FIG. 2. Is a scatter plot generated by applying a non-parametric Wilcoxon ranked-sum test to determine gender differences in gene expression for each of 432 genes on a microarray chip.

FIG. 3. Is a table showing an 11 biomarker subset identified by discriminant analysis, displayed by gene name.

FIG. 4. Is a graphical representation of posterior classification probability of the three phenotypes (ET, RT and normal) using an 11 biomarker gene subset via linear discriminant analysis with leave-one-out cross validation.

FIG. 5. Is a table showing phenotypic binary class prediction using the same algorithm and an 11 biomarker gene subset.

FIG. 6 is a graphical representation of a linear discriminant analysis plot showing the posterior classification probability of each subject by cohort (ET and RT), using an 11 biomarker gene subset based on microarray profiles.

FIG. 7 is a graphical representation of the measurement of platelet samples analyzed by qRT-PCR using oligonucleotide primers specific to each of the 11 genetic biomarkers, the samples coming from a randomly selected subset of 20 subjects (10 ET and 10 RT).

FIG. 8 is a table showing the results of applying discriminant analysis for ET class prediction sub-stratified by the Jak2V⁶¹⁷F allele.

FIG. 9 is a graphical representation of the relative expression of several transcripts detected using a microsphere-based assay.

FIG. 10 is a graphical representation of a correlation coefficient which compares the starting material PRP with the other starting materials.

FIG. 11 is a graphical representation of a correlation coefficient which compares the starting material GFP with the other starting materials.

FIG. 12 is a graphical representation of a correlation coefficient which compares the starting material platelet RNA with the other starting materials.

FIG. 13 is a graphical representation of the detected levels of the various genes over varying platelet concentrations.

FIG. 14 is a graphical representation of the correlation of measured levels between the micro-sphere assay and a microarray assay.

### DETAILED DESCRIPTION OF THE INVENTION

The present inventors have determined a biomarker gene subset that can be used to differentiate three cohorts (Individuals with ET, Individuals with RT and Non-thrombocytotic Individuals (normal)). This is also described in the following publication “Class Prediction Models of Thrombocytosis Using Genetic Biomarkers,” by Dmitri V. Gnatenko et al., BLOOD (2009) (Gnatenko I), which is incorporated herein by reference. The biomarker subset can include identification of the gene expression of a 4 biomarker subset, an 11 biomarker subset, a 15 biomarker subset or variations of the 15 biomarker subset including at least 4 genes. The entire 15 biomarker subset includes the following genes; WASF3, CTNS, HIST1H2AG, ACOT7, LATPM4B, TGFB2, TPM1, H3F3A, APP, NGFRAP1, CLEC1B, HIST1H1A, SRP72, C20orf103 and CRYM.

Linear discriminant analysis with cross-validation was used to identify gene subsets that segregated phenotypes based on microarray profiles. The biomarker gene subset accurately identifies a sample as ET, RT or normal at an accuracy of greater than 85%. Further the present inventors have determined a biomarker gene subset for Jak2-wild type ET. The biomarker gene subset classified Jak2-wild type ET with greater than 85% accuracy. The JAK2V⁶¹⁷F mutation is present in about 60% of all patients with ET, and is presumptive evidence of an ET diagnosis. In embodiments of the present invention, the diagnostic methods can be either nucleic acid-based assays or protein-based assays. That is, the methods can be based on detecting the level of expression of the relevant gene, or based on detecting the level of the expressed protein product, in a hematologic sample taken from a test subject containing platelets.

One embodiment of a method to determine the gene expression profile of thrombocytotic sensitive genes is described herein.

To use non-parametric linear discriminant analysis (NLDA) in determining which disease category a subject's genetic profile is found, two separate groups of subjects were selected. The first group is referred to as the training set, the second group referred to as the independent testing set. The training set is used to measure and analyze gene expression of several genes to determine which genes have a different expression level based on what cohort the subjects are part of in the training set. The independent testing set is used to validate that the differentially expressed genes determined in the training set to differentiate the subject in the independent testing set by cohort. Initially, hematologic samples from subjects in a training set are obtained. These samples can be conventionally obtained by a hypodermic needle, and can consist of whole blood or platelets. Next, the obtained hematologic samples are analyzed with the use of a microarray. The microarray can be an oligonucleotide chip uniquely designed and fabricated for comparative analysis of platelet-expressed genes. This chip can be an Affymetrix HU133A GeneChip. The samples hybridize to individual platelet chips for 12-16 hours and were washed and scanned for quantification of fluorescence intensity.

Next, the expression value for each gene on the microarray was measured. The expression values can be measured by measuring fluorescence intensities. In one embodiment, a Gene Pix 4000B scanner (Molecular Devices, Sunnyvale, Calif.) can be used to measure the fluorescence intensity. Further, analysis was performed to identify differentially expressed genes among three cohorts of thrombocytosis. The three cohorts that are identified are Non-thrombocytotic Individuals (normal subjects), subjects with Essential Thrombocythemia (ET) and subjects with Reactive Thrombocytosis (RT). A subject is identified as being in one of the three cohorts. In one embodiment, this analysis includes a Kruskal-Wallis, non-parametric one-way analysis of variance (ANOVA), followed by the nonparametric Wilcoxon rank-sum test to examine median differences between two independent samples, followed by NLDA with a leave-one-out cross-validation analysis, which develops a statistical classifier designed to categorize and predict clinical phenotypes (ET, RT or normal). This analysis identified differentially expressed genes, and is further described in Example 1 below. 11 of the 15 differentially expressed genes comprise the following; WASF3, CTNS, HIST1H2AG, ACOT7, LATPM4B, TGFB2, TPM1, H3F3A, APP, NGFRAP1 and CLEC1B. The primers of these 11 genes which are used during Quantitative PCR (qPCR) are listed in Table 1 below.

TABLE 1
Primers are shown in 5′-3′ orientation,
F-forward, R-reverse.

| # | Gene name | Primers |
|---|---|---|
| 1 | WASF3 | F: AGGATGGGCTGAAGTTCTATACTG |
| | | R: ATTTTCCTTCTCTCCCACTCTTCT |
| 2 | CTNS | F: CATCTGGAGTACAGGACATAGCTC |
| | | R: TGTGAGGTGGTCTCAAGTACAAAT |
| 3 | HIST1H2AG | F: TAAAATGTCTGCCTCACAGATAGG |
| | | R: TAACCCTCCTTTACAGAAAACTGC |
| 4 | ACOT7 | F: GAGACCGAGGACGAGAAGAAG |
| | | R: CTCTCAATGTGAATTGGGTTTTT |
| 5 | LAPTM4B | F: CTTGTTTGTTGCTGAAATGCTACT |
| | | R: AAAGCAGACTTCTAGGTCCATCAG |
| 6 | TGFB2 | F: GTAAAGTCTTGCAAATGCAGCTAA |
| | | R: ACAAACAGAACACAAACTTCCAAA |
| 7 | TPM1 | F: TTAAACACCTGCTTACCCCTTAAA |
| | | R: GCACACTGTGTTGCTAAACTCTCT |
| 8 | H3F3A | F: AGAATCCACTATGATGGGAAACAT |
| | | R: AAATTCACACACAAATGAAAATGG |
| 9 | APP | F: CAACCTACAAGTTCTTTGAGCAGA |
| | | R: TCAAGTTCAGGCATCTACTTGTGT |
| 10 | NGFRAP1 | F: GGAAATATTCATGGAGGAGATGAG |
| | | R: AAAATGCAAGGAAAAAGAAAACAG |
| 11 | CLEC1B | F: AAACCAAATAGGAAACTCAAATGC |
| | | R: ATTAAACCCATTTCAAGGTGAGAA |

The other 4 of the 15 differentially expressed genes comprise the following; HIST1H1A, SRP72, C20orf103 and CRYM. The primers of these 4 genes which are used during Quantitative PCR (qPCR) are listed in Table 2 below. This is further described in Example 4 below.

TABLE 2
Primers are shown in 5′-3′ orientation,
F-forward, R-reverse.

| | | |
|---|---|---|
| 12 | HIST1H1A | F: TCTACTGCAGAAAAGAAGACGAGA |
| | HIST1H2BK | R: GCAAAAGAGGAGATGGGTAATGTA |
| 13 | SRP72 | F: TAGGGAAACATCTTCCTCAAAGTC |
| | | R: CTGTTTAAATCCTGGGAAACCTTA |
| 14 | C20orf103 | F: CCTATACAGTTGTCAATGCACACA |
| | | R: ACAGGAAGAAGTCCAAACAACTCT |
| 15 | CRYM: | F: GAATATTGCTGCTGGTTCTCATAA |
| | | R: TCAGGGAAATATAAAGGGAAATGA |

qPCR was performed to validate these gene biomarkers using the samples of the training set. The training data is used to directly classify the testing set using NLDA, as described in the next step. The group level sensitivity and specificity of the classification as well as obtaining the posterior classification probability at each individual subject level is done for the independent testing set. These posterior probabilities show how likely each subject belongs to each disease category and sum to 1 across all categories.

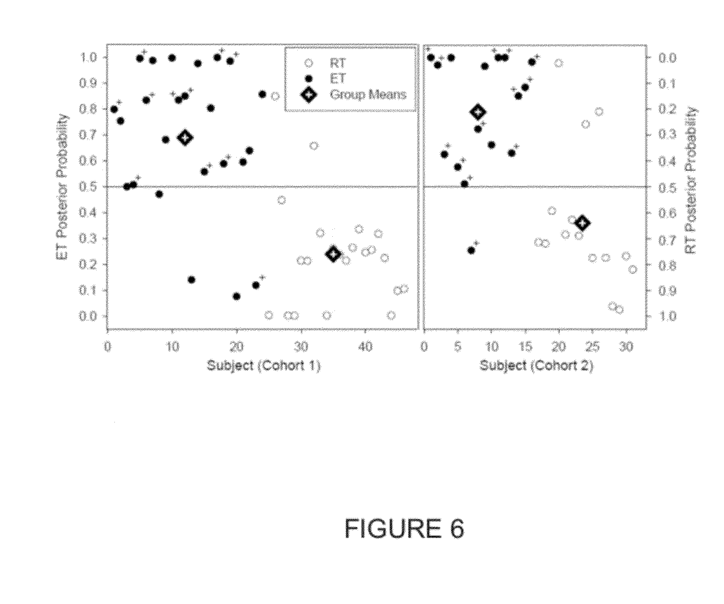

The next step in the method to determine the gene expression profile of thrombocytotic sensitive genes includes obtaining hematologic samples from subjects in an independent testing set for validation of the gene expression profile identified in the training set. These samples can be conventionally obtained by a hypodermic needle, and can consist of whole blood or platelets. Following this step, the identity of the differentially expressed genes among the three cohorts of thrombocytosis is validated. In one embodiment, this validation is determined by use of qPCR, which measures the gene biomarkers of the samples in the independent testing set. The training data was used to directly classify the independent testing set using NLDA. The group level sensitivity and specificity of the classification as well as the posterior classification probability at each individual subject level were obtained for the independent testing set, and are shown in FIG. 6 below.

These differentially expressed genes can also be used to classify a subject as being Jak2-wild type. The JAK2V⁶¹⁷F mutation is present in about 60% of all patients with ET, and is presumptive evidence of an ET diagnosis.

The method to determine the gene expression profile of thrombocytotic sensitive genes is further described in Examples 1 and 3 below.

Another method to distinguish thrombocytosis cohorts is described herein. The method to distinguish thrombocytosis cohorts includes the first step of obtaining a hematologic sample from a subject. These samples can be conventionally obtained by a hypodermic needle, and can consist of whole blood or platelets. Next, the gene expression of a biomarker subset is determined. In one embodiment, gene expression profiles can be determined using an oligonucleotide chip uniquely designed and fabricated for comparative analysis of platelet-expressed genes. The expression values can be measured by measuring fluorescence intensities. In one embodiment, a Gene Pix 4000B scanner (Molecular Devices, Sunnyvale, Calif.) can be used to measure the fluorescence intensity.

Next the gene expression of the biomarker subset is analyzed. The statistical technique used to identify differently expressed genes among the three cohorts can be stepwise linear discriminant analysis. Stepwise linear discriminant analysis is a statistical technique to classify objects into mutually exclusive and exhaustive groups based on a set of measurable features. Kernel based nonparametric linear discriminant analysis (NLDA) is used to categorize a subject's genetic profile into a disease category because the genetic data measured is not all normally distributed. This analysis can be used to classify subjects into different disease categories based on a subject's platelet genetic profile, which is the next step of the method to distinguish thrombocytosis cohorts. The gene expression of a biomarker subset is used to classify a subject into one of three cohorts, ET, RT and normal. The biomarker subset can include identification of the gene expression of a 4 biomarker subset, an 11 biomarker subset, a 15 biomarker subset or variations of the 15 biomarker subset including at least 4 genes. The entire 15 biomarker subset includes the following genes; WASF3, CTNS, HIST1H2AG, ACOT7, LATPM4B, TGFB2, TPM1, H3F3A, APP, NGFRAP1, CLEC1B, HIST1H1A, SRP72, C20orf103 and CRYM.

The next step of the method to distinguish thrombocytosis cohorts is classifying the subject into one cohort. There are 3 cohorts, ET, RT and normal. In one embodiment, classification posterior probability is calculated for the subject. The classification posterior probability is a representation of the probability that a subject belongs to one of three cohorts, ET, RT or normal. The classification is based on the joint distribution of biomarkers. For exemplary purposes, if a sample from a subject is tested, and the posterior possibilities are calculated as 0.2 ET, 0.1 RT and 0.7 normal, the subject would be classified as normal. For a binary decision, the subject is classified to the phenotype with the highest probability. The gene expression values in a training set of subjects are used to determine the unknown parameters in the density function, which are used as the inputs into a statistical program, for example SAS® Version 9.12 (SAS Institute Inc. Cary, N.C., USA) PROC DISCRIM with Method=NONPAR and Pool=YES. The biomarker expression value of a test subject is taken into account to determine the density function to generate the posterior classification probabilities for the three cohorts, ET, RT and normal using Bayes' theorem in conjunction with the following formulas. At least 4 genes of the 15 biomarker subset can be used to determine the subject's cohort and whether or not the subject has the JAK2V⁶¹⁷F mutation. In another embodiment, at least 5, at least 6, at least 7, at least 8, at least 9, at least 10, at least 11, at least 12, at least 13, at least 14 or all 15 genes of the 15 biomarker subset can be used to determine the subject's cohort and whether or not the subject has the JAK2V⁶¹⁷F mutation. The expression data found in the GEO database, in MIAME-compliant form, reported under National Center for Biotechnology Information (NCBI) accession #15670131 (Series GSE12295) was used.

PROC DISCRIM computes p(t/x), the probability of x belonging to group t, by applying Bayes' theorem:

$$p ( t \vert x ) = \frac{{q}_{i} {f}_{t} ( x )}{f ( x )}$$

PROC DISCRIM partitions a p dimensional vector space into regions Rₜ, where the region Rₜ is the subspace containing all p-dimensional vectors y such that p(t/y) is the largest among all groups. An observation is classified as coming from group t if it lies in region Rₜ.

The non-parametric method does not give explicit linear discriminant functions, but only calculate the posterior density that a subject belongs to each group t based on his/her gene biomarker set expression vector x.

### Normal Kernel (with Mean Zero, Variance r²Vₜ)

$${K}_{t} ( z ) = \frac{1}{{c}_{0} ( t )} exp ( - \frac{1}{2 {r}^{2}} {z}^{′} {V}_{t}^{- 1} z )$$

$$where {c}_{0} ( t ) = {( 2 π )}^{\frac{p}{2}} {r}^{p} \vert {V}_{t} {\vert}^{\frac{1}{2}} .$$

Here, Vₜ is the pooled correlation matrix because POOL=YES in PROC DISCRIM.
The group t density at x is estimated by

$${f}_{t} ( x ) = \frac{1}{{n}_{t}} {\sum }_{y} {K}_{t} ( x - y )$$

x a p-dimensional vector containing the quantitative variables of an observation
Sₚ, the pooled covariance matrix
t a subscript to distinguish the groups
nₜ the number of training set observations in group t

Application of the Bayes' Theorem provides:

$$p ( t \vert x ) = \frac{{q}_{t} {f}_{t} ( x )}{f ( x )}$$

where f(x)=Σᵤqᵤfᵤ(x) is the estimated unconditional density.
   qₜ t the prior probability of membership in group t
   p(t|x) the posterior probability of an observation x belonging to group t
   fₜ the probability density function for group t
   fₜ(x) the group-specific density estimate at x from group t
   f(x) Σₜqₜfₜ(x), the estimated unconditional density at x

The detailed algorithm can be found in Rosenblatt, M., 1956. Remarks on Some Nonparametric Estimates of Density Function. Ann. Math. Statist. 27930, 832-837, which is incorporated herein by reference. This classification is further described below.

In a further embodiment, the invention provides an ability to determine whether a subject has ET, and whether a subject has RT and whether a subject is normal (i.e. does not have either ET or RT). In this embodiment the method to determine comprises obtaining a hematologic sample from a subject, determining and measuring the gene expression of a set of biomarkers and classifying the subject into one of three cohorts based on the measurement of the gene expression of the set of biomarkers.

In another aspect of the invention, a kit is provided to distinguish thrombocytosis cohorts. The kit comprises a hematologic sampler, this hematologic sampler can be a hypodermic needle. The kit further comprises reagents. These reagents comprise the following non-limiting examples; reverse transcriptase, a reverse transcriptase primer, corresponding PCR primer set, a thermostable DNA polymerase, and a suitable detection reagent, such as, without limitation, a scorpion probe, a probe for fluorescent hydrolysis probe assay, a molecular beacon probe, a single dye primer or a fluorescent dye specific to double-stranded DNA, such as ethidium bromide. The kit further comprises one or more reaction vessels. These reaction vessels can be, for example, test tubes or beakers. The kit further comprises various gene expression profile platforms adapted to express biomarker subsets.

In one embodiment, the gene expression profile platform can be a microarray, and more specifically an oligonucleotide chip, which is fabricated and designed for comparative analysis of platelet-expressed genes. One example of a chip is an Affymetrix HU133A GeneChip.

In another embodiment, the gene expression profile platform can be a platelet qRT-PCR. If the gene expression profile platform is a qRT-PCR, the kit can also include primers for amplifying DNA of a hematologic sample by PCR. These primers include, but are not limited to, primers for genes WASF3, CTNS, HIST1H2AG, ACOT7, LAPTM4B, TGFB2, TPM1, H3F3A, APP, NGFRAP1, CLEC1B, HIST1HIA, SRP72, C20orf103, and CRYM and combinations of primers for these genes. These primers can be seen in Tables 1 and 2 above. In this embodiment, the kit can also include platelet isolators, including high quality platelet RNA such as TRIzol®.

In another embodiment, the gene expression profile can be a multiplex bead based assay configured to quantitatively measure one or several mRNA transcript levels. Such bead assays are available from Panomics™.

The kit can further include an analyzer to measure the gene expression profile platform. This analyzer can be any analyzer capable of measuring gene expressions including a GenePix 4000B scanner by Molecular Devices or a BioPlex reader by Bio-Rad, Hercules, Calif.

The kit can further comprise primers for amplifying DNA of a hematologic sample by PCR. The corresponding primer set for the biomarker gene subset can be seen in Tables 1 and 2, and are listed in the 5′-3′ orientation, with F being for forward and R being for reverse. Other primers for amplifying the biomarkers via PCR can be designed using tools well known by those in the art.

The present invention is further illustrated by the following non-limiting examples.

### Example 1

### Patient Recruitment

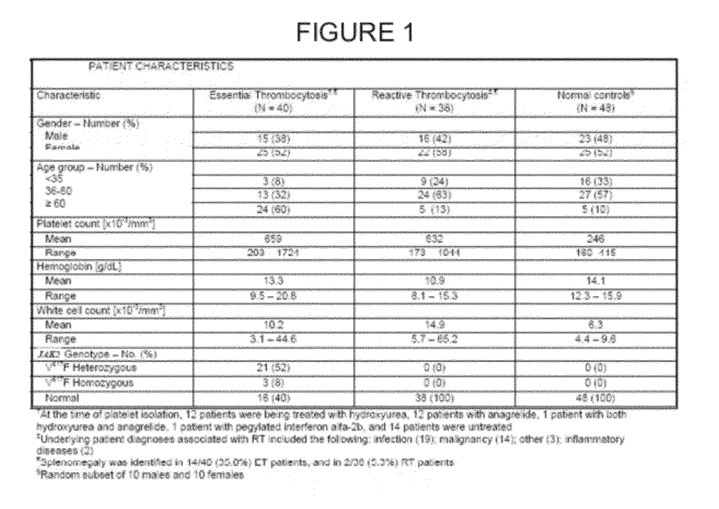

To determine the correct biomarker gene set, expression of which will distinguish ET and RT, samples of blood were taken from several individuals. Samples were taken from 95 subjects who were randomly enrolled from a larger pool of patients referred for evaluation of thrombocytosis. All subjects provided informed consent for an IRB (Institutional Review Board)-approved protocol completed in conjunction with the Stony Brook University General Clinical Research Center. Both sex- and age-distribution paralleled prevalence figures for ET with a M:F ration of 1:2; age at diagnosis ranged from 23-78 years. Platelet counts at the time of blood isolation ranged from normal levels of about 246,000/μL, to 1,724,000/μL; utilization of platelet-lowering drugs was recorded for individual patients at the time of platelet isolation and purification, as can be seen in FIG. 1.

### Molecular Studies

Leukocytes and gel-filtered platelets were isolated from peripheral blood (20 mL) as described in Gnatenko D V, Dunn J J, McCorkle S R, Weissmann D, Perrotta P L, Bahou W F. Transcript Profiling of Human Platelets using Micromay and Serial Analysis of Gene Expression. Blood 2003; 101(6):2285-93(Gnatenko II), which is incorporated herein by reference. The final platelet-enriched product contained no more than 3-5 leukocytes per 1×10⁵ platelets. High-quality platelet RNA was isolated using Trizol, and platelet mRNA quantification and integrity were established using an Agilent 2100 Bioanalyzer; mean platelet RNA concentrations among the three cohorts were comparable, ranging from ˜0.3-1.0 fg/platelet. High molecular weight DNA was used as the source for genomic JAK2V⁶¹⁷F (exon 12, 1849^{G→T} transversion) genotyping, while platelet mRNA was used for cellular genotyping. Mutational screening was completed using both pyrosequence and dideoxy sequence analyses of PCR-amplified fragments. Samples were defined as JAK2V⁶¹⁷F-positive if the mutant allele was detected in greater than 5% of the nucleic acid pool.

Confirmatory studies of platelet gene expression were established using fluorescence-based real-time PCR. Oligonucleotide primer pairs were generated using Primer3 software, designed to generate 200±1 base pair PCR products at the same annealing temperature. mRNA levels were quantified using real-time fluorometric analysis, and relative mRNA abundance was determined from triplicate assays using the comparative threshold cycle number (Δ-Ct method).

### Chip Design and Manufacture

Gene expression profiles were determined using an oligonucleotide chip uniquely designed and fabricated for comparative analysis of platelet-expressed genes. The gene list was generated using microarray profiles from a cohort of normal (N=5) and ET (N=6) platelet mRNA's hybridized to the Affymetrix HU133A GeneChip. Leukocyte RNA from three normal patients was used to delineate leukocyte gene expression profiles. Finalized, custom spotted microarrays contained 432 platelet-expressed and 43 leukocyte-restricted genes which co-segregated by cell-type (platelet vs. leukocyte). Arabidopsis probe elements were included for normalization controls and as quantitative measures of inter- and intra-slide variability; 70-mer oligonucleotides were synthesized based on the Ensemble Human 13.31 Database; all probe-sets were spotted in quadruplicate to provide replicates and statistical robustness.

### Gene Expression Analysis

Platelet gene profiling was completed using a template-switching mechanism to optimize amplification from low-abundance mRNA's. Initially, 20 ng of purified platelet or human reference RNA (Stratagene) was supplemented with a fixed amount of Arabidopsis mRNA to provide internal standards for hybridization and normalization. Chimeric DNA/RNA amplification and labeling was completed using the Ovation Aminoallyl system from NuGen Technologies, providing for 4-6 μg of cDNA/sample. cDNA solutions were vacuum-dried and coupled to Cy3 (human reference RNA) or Cy5 (patient RNA) dyes from Amersham Biosciences, and stoichiometrically equivalent mixes were hybridized to platelet chips prior to gene quantification using a Gene Pix 4000B scanner (Molecular Devices). All microarray data were submitted to the GEO database in MIAME-compliant form, reported under National Center for Biotechnology Information (NCBI) accession #15670131 (Series GSE12295). Initial data processing (gridding, technical spot analysis, etc.) was completed using GenePix Pro software. After rigorous inspection to exclude spotting irregularities, raw Cy3:Cy5 ratios were quantified for individual genes. Reproducibility of microarray profiles using biological replicates from healthy donors produced a Spearman correlation coefficient of 0.93-0.95.

### Bioinformatics and Statistical Analyses

Microarray data were analyzed and visualized using GeneSpring (Silicon Genetics) or a custom software product. Expression data were sequentially normalized by spot, by gene, and by chip essentially as previously described, followed by a moderate filtering step to maximize our ability to identify differentially-expressed genes. Genes with fluorescence intensities <10 in more than 70% of the probes were excluded from further analysis. For each gene, the four ratios were averaged and log₂-transformed prior to data analysis. The Kruskal-Wallis, non-parametric one-way analysis of variance (ANOVA) was performed to identify differentially-expressed genes among the three cohorts (ET, RT, Non-thrombocytotic). The nonparametric Wilcoxon rank-sum test was used to examine median differences between two independent samples. This included gender effects, the comparison between ET and RT subjects, as well as comparison within ET subjects by Jak2 genotype using either microarray or qRT-PCR data. The significance level is set at 0.05 (two-sided) unless otherwise specified.

Stepwise discriminant analysis was used to identify an initial biomarker subset that separated class on the basis of microarray data. The fidelity of the genetic biomarker subsets as class prediction tools was established using non-parametric linear discriminant analysis with a leave-one-out-cross-validation analysis. Posterior classification probability for each subject was derived and the binary decision was made for group assignment based on subject highest probability. As part of the confirmatory studies, the same biomarker set using the microarray data was applied to the qRT-PCR data, and fidelity established using non-parametric linear discriminant analysis with a leave-one-out-cross-validation analysis. This same biomarker identification and validation procedure was applied both for separation of ET vs. RT, and for substratification of ET by Jak2 genotype (Jak2V⁶¹⁷F vs. wild-type alleles).

### Results Discussion

Out of a total of 95 subjects (ET [N=24]; RT [N=23]; normal [N=48]), the mean platelet counts for ET and RT subjects were not statistically different. At the time of platelet collection 4/24 ET and 1/23 RT patients had normal platelet counts reflecting an effect of medication for ET or thrombocytotic resolution for RT. Of the ET patients, 46% were heterozygous for the Jak2V⁶¹⁷F mutant (GT) allele, while a smaller fraction (8%) was found to be homozygous for the mutation (TT). No RT or non-thrombocytotic patients harbored the Jak2V⁶¹⁷F mutation.

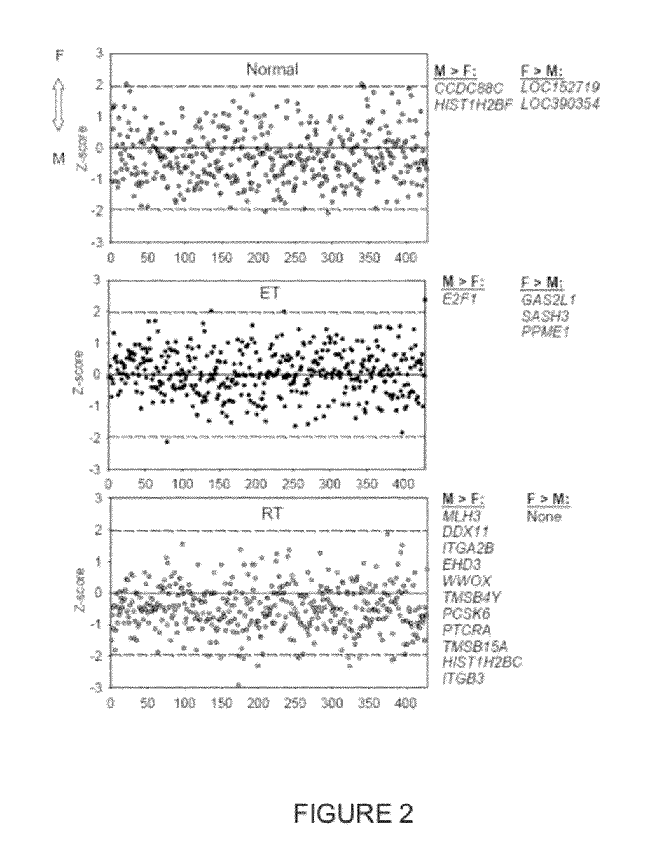

Based on evidence of genetic differences between normal and ET platelets, a platelet-focused chip was fabricated. Initially, in seeking to exclude gender effects among the three cohorts a Wilcoxon rank-sum test was performed for each of the 423 genes on the array. For both non-thrombocytotic (normal) and ET cohorts, the preponderance of the genes were equally distributed within the 95% Confidence Interval (CI), with only four of the genes in either group demonstrating any gender effect. In normal patients, two genes displayed greater expression in males (MBOAT2 and H2BF), while two other genes were differentially-weighted towards females (LOC152719 and LOC390354), as seen in the top section of FIG. 2. In ET platelets, a single gene (E2F1) was male-skewed, while three genes (GAS2L1, CXORF9, and PPME1) were female-biased, as seen in the middle section of FIG. 2. In contrast, gender effects were more prominent in the RT cohort, with 12 genes falling outside the 95% CI, all of which demonstrated male-skewed gene expression differences, as seen in the bottom section of FIG. 2. Two of these 12 genes (ITGA2B and ITGB3) encode the major polypeptide subunits of the platelet glycoprotein IIB/IOIIA (α_{IIb}/β_{III}) integrin, suggesting that the molecular mechanisms that control gene expression of the heterodimeric receptor complex are concordantly regulated by gender during situations associated with RT.

### Delineation of a Genetic Biomarker Subset for Discriminant Analysis

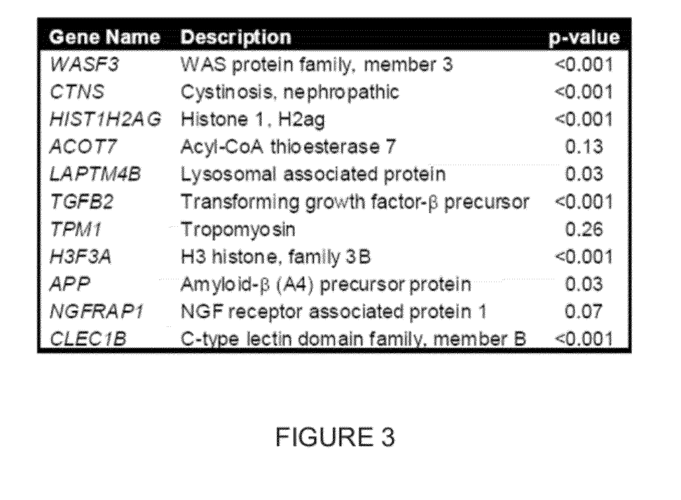

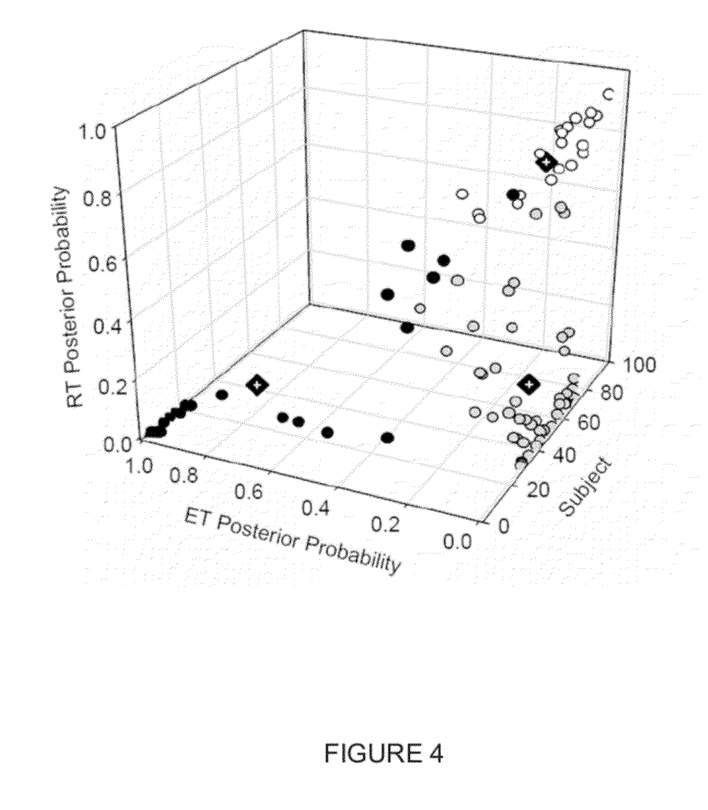

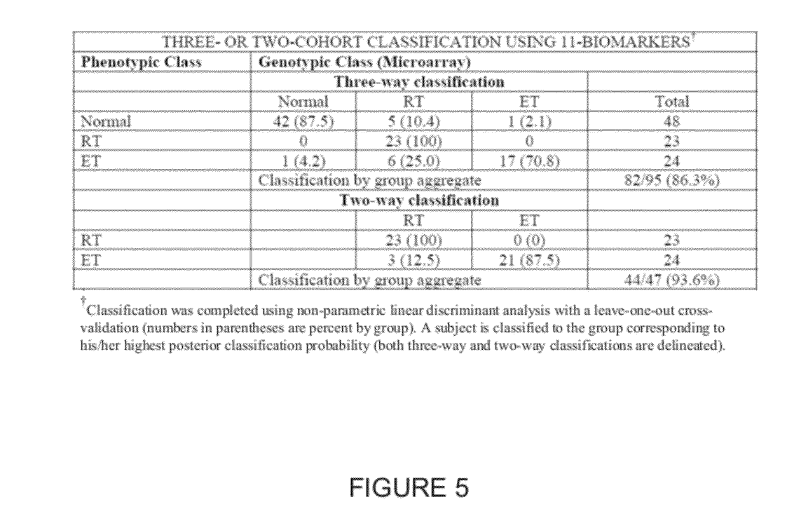

Of the genes on the microarray chip, 267 displayed expression values that were significantly different among the three groups (p<0.05), as established using the Kruskal-Wallis non-parametric one-way ANOVA. Among this subset, 148 genes were found to be significantly different between RT and ET cohorts using the Wilcoxon rank sum test. Stepwise LDA identified an 11-biomarker subset that segregated the three phenotypic cohorts (ET vs. RT vs. non-thrombocytotic) as listed in FIG. 3. The utility of the initial 11-biomaker subset to predict class was confirmed using a non-parametric linear discriminant analysis with a leave-one-out-cross-validation analysis, in which each case is classified by the profiles derived from all cases excluding that case. This approach confirmed the generalizability of the statistical classifier (i.e. its performance on previously unseen data) by using the available data as both training and test data, thereby providing an unbiased estimate of class prediction. The posterior classification probabilities applied in a binary decision model using this gene-set for 3-cohort analysis confirmed that 82/95 (86.3%) of all patients could be correctly classified, as seen in FIGS. 4 and 5.

### Example 2

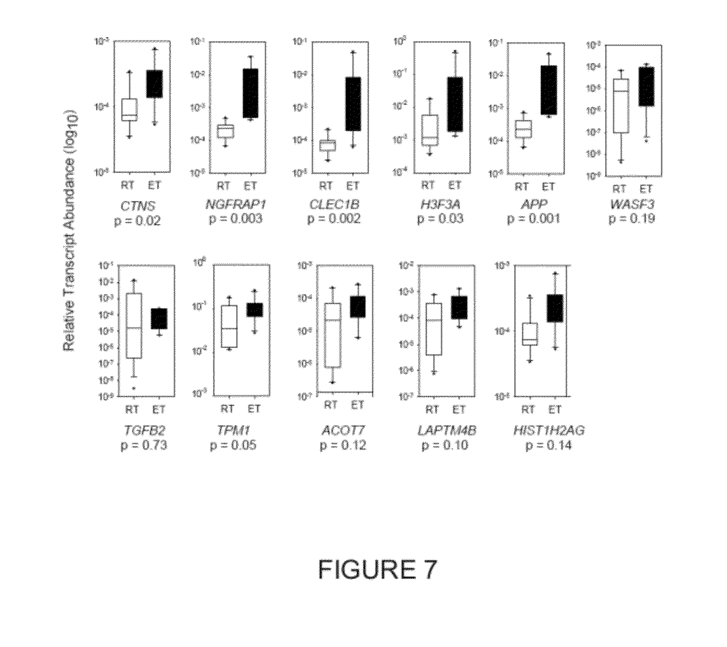

An 11-biomarker subset was also used to classify 2-cohorts (ET from RT) as compared with the 3-cohort comparison from Example 1. Two-cohort LDA confirmed that ET and RT profiles segregated by class give an overall accuracy rate of 93.6%, as can be seen in FIGS. 6 and 7.

### Example 3

As an additional validation of an 11-member gene subset to discriminate between ET and RT cohorts, platelet gene profiles were re-analyzed using a confirmatory platform. Oligonucleotide primers were generate to the 11 biomarker gene set, and completed qRT-PCR for a randomly selected subset of 10 patients in each cohort. Six of the biomarkers were found to have significantly different median expression levels between ET and RT cohorts via qRT-PCR at p<0.05 (CTNS, NGFRAP1, CLEC1B, H3F3A, APP and TMP1), as seen in the top portion of FIG. 7. These confirmatory results show that ET and RT profiles are genetically distinct. As shown in FIG. 7, binary class prediction using either microarray or qRT-PCR data alone gave accurate results.

### Example 4

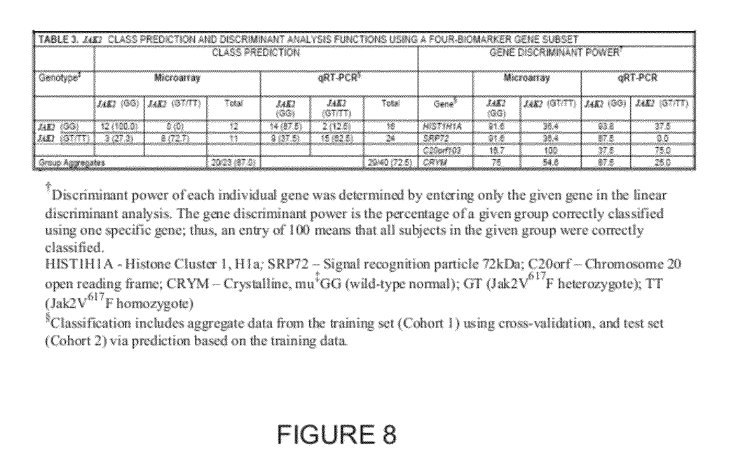

A discriminant and validation analysis for ET class prediction sub-stratified by the Jak2V⁶¹⁷F allele was also conducted. While the presence of the Jak2V⁶¹⁷F mutation is strong presumptive evidence for the diagnosis of ET, as shown below in FIG. 1, the absence of the mutation occurs in up to 40% of ET patients. Stepwise discriminant analysis based on the microarray data alone resulted in a 4-member subset comprised of genes HIST1, SRP72, C20orf103 and CRYM. The primers of these 4 genes which are used during qPCR are listed in Table 2 below.—LDA with cross-validation based on the microarray data alone confirmed that 87% of patients were correctly classified, as seen in FIG. 8. Comparable results are provided when using qRT-PCR as a validation platform in which more than 90% of Jak2-wild type subjects were correctly classified. The overall correct classification rate using confirmatory qRT-PCR is 73.9%, as can be seen in FIG. 8.

### Example 5

When the gene expression profile is a bead based assay, the assay can be configured to quantitatively measure one or several mRNA transcript levels. Such bead based assays are available from Panomics™ and determine the level of gene expression profile of various genes. The bead based assay is designed to quantify various mRNA transcript levels from various genes. The bead based assay is conducted without the need for RNA purification, reverse transcription or amplification.

Bead based assays can target several RNA transcripts and several gene expressions in a single vessel in a single test from a cultured cell or whole blood lysate. The bead based assay operates similarly to other bead based assays produced by Luminex0, among others. Initially, the specific transcripts desired to be measured are chosen. Once these transcripts are chosen, each of the beads is coated with an oligonucleotide specific to the transcript chosen.

Each bead is a 5.6 micron polystyrene microsphere coated and filled with specific dye mixtures.

The beads are analyzed in a flow cytometry based instrument which utilizes lasers and a detector to measure the spectral signature of each bead. Based on the specific dye mixture of the bead and each dye mixture's unique spectral signature, the flow cytometry based instrument determines what reagent is coated on the bead which thereby determines the gene expression level of the specific gene.

In this example, a Luminex0 based assay, specifically a Plex set 11032 Catalog #311032 assay from Panomics™ was used. The bead or microsphere-based multiplex gene expression analysis platform was developed for comparative transcript profiling using either intact cells or total cellular RNA. This branched DNA (bDNA) gene detection system is a sandwich nucleic hybridization assay that quantifies mRNA directly from cellular lysates by amplifying the reporter signal rather than target transcripts. This particular assay was used to show that the microsphere based assay is capable of detecting and accurately measuring various genes across a large range. The accuracy of the measurements was verified, as further described below, through a comparison of expression levels with a known microarray.

Seventeen transcripts were chosen to represent gene abundances at extreme ranges in human platelets. Prior research has demonstrated that the dynamic range of expression abundance of these transcripts spanned nearly 4-logs. This prior research has been quantified and described in Gnatenko D V, Cupit L D, Huang E C, Dhundale A, Perrotta P L, Bahou W F. “Platelets Express Steroidogenic 17β-Hydroxysteroid Dehydrogenases,” Thromb Haemost. August 2005; 94(2):412-421(Gnatenko III), the content of which is incorporated herein by reference. This research indicates that the 17 transcripts which were analyzed in the bead based assay represent a wide spectrum of gene expression as confirmed by microarray analysis. Further, the data gathered in Gnatenko II and Gnatenko III was used as a comparison to determine whether the bead based assay described below is accurate over the range of values determined by the microarray for these particular transcripts. The discussion of this comparison is further described below.

Three types of oligonucleotides were generated for each of the 17 mRNAs, collectively designed to optimize (i) mRNA capture, (ii) signal amplification, and (iii) mRNA stabilization. All probes were uniquely designed for ˜500-base region of mRNA. Oligonucleotides were covalently linked to the microspheres, thereby providing unique microsphere-specific signatures for each transcript. For all microspheres and probe sets, coupling efficiencies were optimized and quality-controlled to minimize nonspecific hybridizations. The coupling of oligonucleotides to the microspheres is described in Zheng Z, Luo Y, McMaster G K. “Sensitive and Quantitative Measurement of Gene Expression Directly From a Small Amount of Whole Blood.” Clin. Chem. July 2006; 52(7):1294-1302, the content of which is incorporated herein by reference.

### Platelet and RNA Isolation for Microsphere-Based Platelet Profiling

In this example, two 20 mL samples of whole blood were collected. The first 20 mL sample was underwent platelet isolation. Platelet-rich plasma (PRP) was prepared by centrifugation for 3.5 min at 1,800 rpm (˜700 g) at 25° C.; The upper 9/10 of PRP (˜5 mL total) was subsequently harvested and supplemented with 0.1 μM prostaglandin E1 (PGE₁) and 10 mM EDTA, loaded onto a Sepharose 2B column equilibrated with Hepes-buffered modified Tyrodes (HBMT: 10 mM HEPES [N²-hydroxyethylpiperazine-N′-2ethanesulfonic acid], pH 7.45; 137 mM NaCl; 2.7 mM KCl; 0.4 mM NaH₂PO₄; 12 mM NaHCO₃; 0.2% BSA; 0.1% dextrose), in the presence of 0.1 μM prostaglandin E1 (PGE₁) and 10 mM EDTA. Gel-filtered platelets (GFP) were harvested after the column.

The second 20 mL sample was divided in two parts, each part used to produce either PRP or GFP platelet fractions. Transcript quantification was achieved using intact platelets (PRP or GFP) and total platelet RNA in parallel.

### Platelet Transcript Profiling Using Microspheres

Platelet lysates were prepared using a cell lysis buffer (Panomics™, Fremont, Calif.) supplemented with 50 μg/μL proteinase K, followed by a 30 minute incubation at 65° C. After serial dilution (1:3 and 1:9) into the same lysis buffer, individual 80 μL aliquots were captured onto microspheres (2000 microspheres of each type per assay) in a 100 μL reaction. For transcript profiling from intact platelets, the following number of platelets were used: [GFP ˜5×10⁷, 16×10⁷, and 46×10⁷ platelets; PRP ˜6×10⁷, 19×10⁷, and 59×10⁷ platelets.] For all experiments, hybridizations were completed in triplicate. Comparative analysis of total RNA was completed in the identical manner, using platelet, leukocyte, human erythroleukemia (HEL) cells or COS-1 total RNA as controls.

After sealing individual wells, hybridizations were allowed to proceed for 16-18 hours overnight at 54° C. Following the overnight capture of the target mRNAs, microspheres were transferred onto 0.45 μm filters (Millipore™, Billerica, Mass.), washed, and sequentially hybridized at 50° C. with the bDNA amplifier and 5′-dT(biotin)-conjugated label probes. Unbound material was washed from microspheres using a vacuum manifold and 0.1×SSC/0.03% lithium lauryl sulfate, followed by 30-minute incubation at 25° C. using streptavidin-conjugated R-phycoerythrin (SAPE). The microspheres were washed to remove unbound SAPE. The spectral signature of each bead was measured using a BioPlex reader (Bio-Rad™, Hercules, Calif.) calibrated to the high sensitivity mode.

### Data Analysis

Relative transcript abundance for each individual gene was established using mean fluorescent intensity (MFI), calculated from fluorescent measurement of 100 microspheres per transcript. The identity (ID) of each target gene was linked to a type of microsphere with a specific spectral signature by design. During the experiment, individual microsphere identifiers were read by the instrument in tandem with quantitative spectral signals, thereby providing simultaneous read-outs of gene ID and transcript abundance. Background signals were established in the absence of target RNAs, and were subtracted from signals derived in the presence of RNAs. The sensitivity of the assay for individual target RNAs was determined by measuring the limit of detection, empirically defined as the target concentration at which the signal is three standard deviations above background. For all experiments, statistical significance was determined by analysis of variance (1- or 2-way ANOVA), while correlation coefficients were established using regression analysis. For all biological comparisons, p<0.05 was used to establish statistical significance.

Comparative Multiplex Gene Expression Analysis of Distinct Platelet Fractions—PRP, GFP and Platelet RNA—Demonstrate Similar Transcript Profiles for 17 Genes

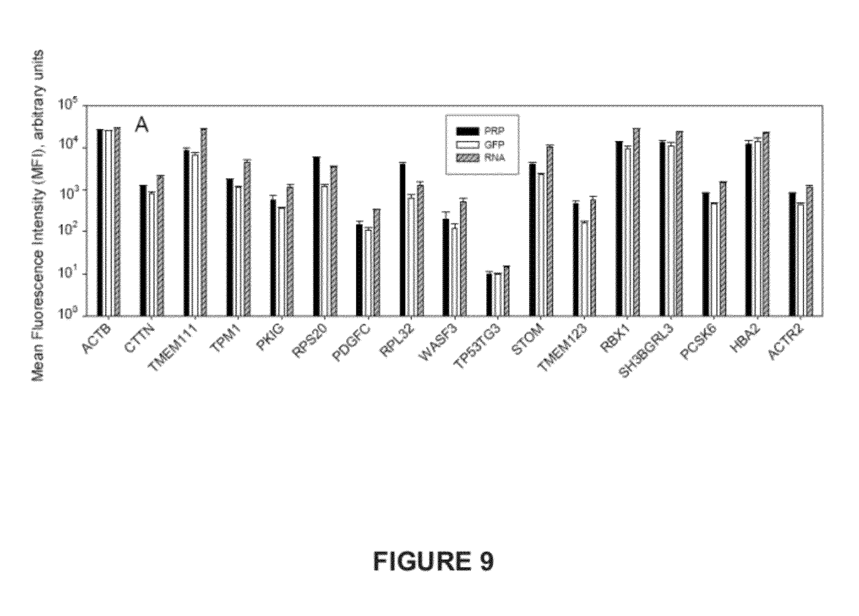

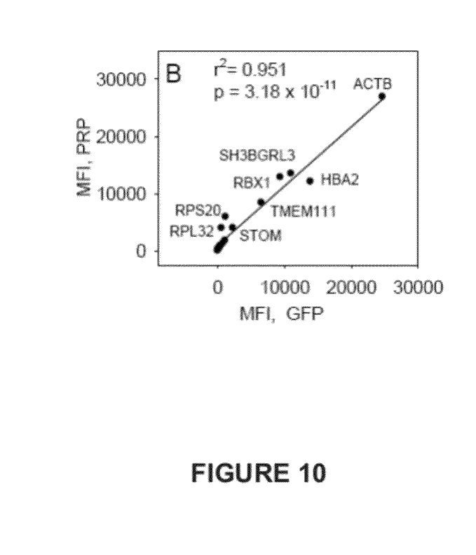

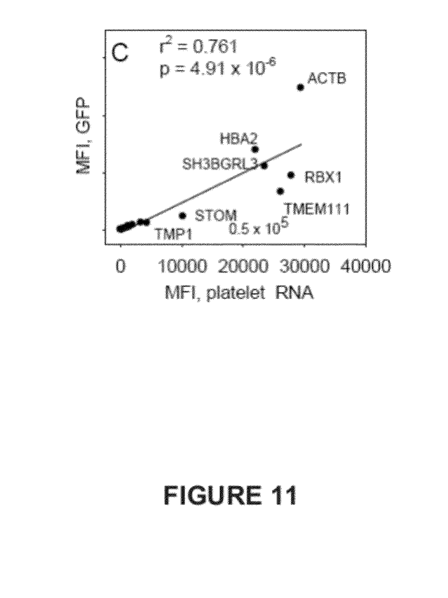

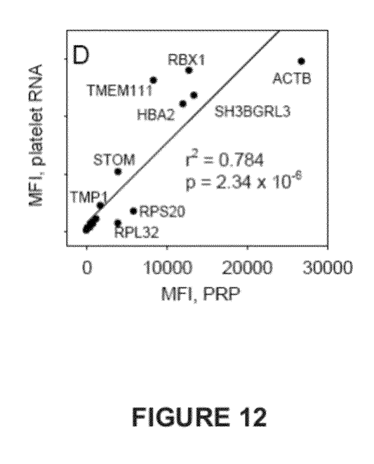

Transcript abundance in two distinct platelet purification fractions (PRP and GFP) and in platelet RNA, purified from the identical GFP fraction was studied. GFP or PRP transcript quantifications were completed using cells lysed in vitro. Transcript analysis of purified platelet RNA was completed on the same 96-well plate in parallel to minimize error. Despite the broad range of relative expression, all transcripts were detected using the microsphere-based multiplexing, as can be seen in FIG. 9. Correlation coefficients comparing each of the starting materials (pure platelet RNA, GFP, PRP) show a good accuracy, as can be seen in FIGS. 10-12. Overall, the standard errors were quite small, demonstrating high reproducibility for both high- and low-abundant transcripts using any of the RNA sources.

### Microsphere-Based Technology Requires Few Platelets

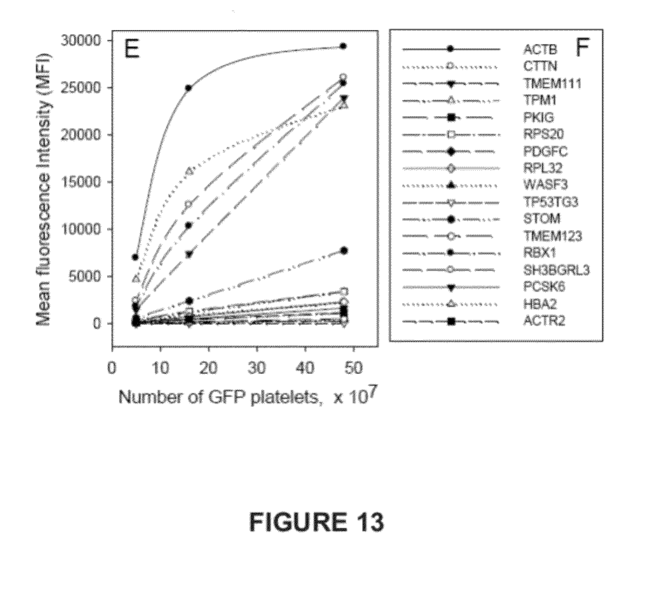

To address the sensitivity of transcript profiling using the microspheres, the analysis using varying amounts of GFP was repeated as can be seen in FIG. 13. Signals for 16/17 transcripts were reliably detected from as few as 5×10⁷ platelets (a platelet mass typically found in ˜100 μL of blood). For two transcripts—ACTB and HBA2—the fluorescent signal reached a plateau due to early microsphere saturation. The remaining 15 transcript curves demonstrated good linearity, suggesting accurate detection over varying platelet numbers. This data also suggests that the optimal number of platelets for microsphere-based quantification of the 17-gene dataset is between 1.5-2×10⁸ platelets per well, based on signal intensity and saturation plateau.

### Validation of Microsphere-Based Transcript Profiling

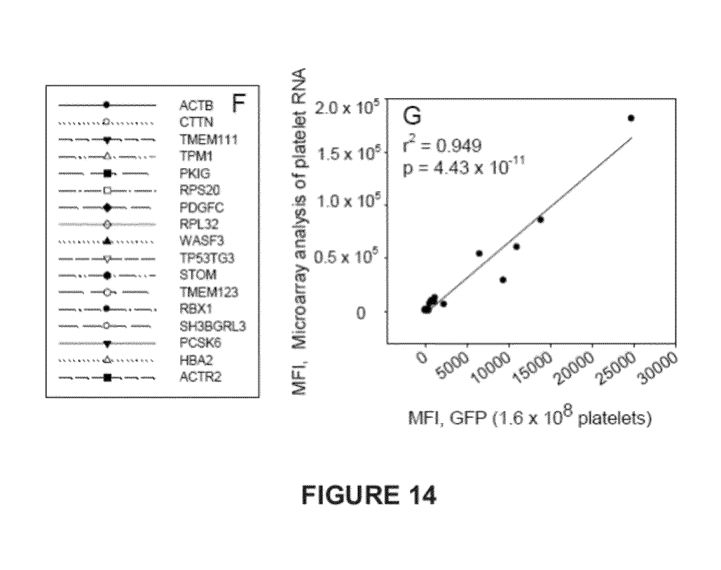

To validate expression levels, transcript profiles obtained from microsphere based multiplex analysis of 1.6×10⁸ GFP platelets were compared to a normal platelet transcriptome database comprised of 5 highly purified apheresis normal platelet Affymetrix™ microarrays, the microarrays described in Gnatenko II and Gnatenko III. Regression analysis demonstrated good concordance between the two platforms—microarray and microsphere-based assay, as can be seen in FIG. 14. The high correlation (r²=0.949, p<1×10⁻¹⁰) reaffirms the accuracy of microsphere-based transcript profiling utilizing low numbers of intact platelets.

What is claimed is:

1. A method to determine the gene expression profile of genes, the method comprising:
obtaining hematologic samples from subjects in a training set;
analyzing the obtained hematologic samples with a microarray;
measuring the expression values of each gene on the microarray;
performing analysis to identify a biomarker subset of differentially expressed genes in the training set among three cohorts of thrombocytosis;
obtaining hematologic samples from subjects in an independent testing set; and
validating the identity of the differentially expressed genes in the independent testing set among the three cohorts of thrombocytosis.

2. The method of claim 1 wherein 4 differentially expressed genes are identified.

3. The method of claim 1 wherein 11 differentially expressed genes are identified.

4. The method of claim 1 wherein 15 differentially expressed genes are identified.

5. The method of claim 1 wherein 4-15 differentially expressed genes are identified.

6. The method of claim 5 wherein the biomarker subset comprises at least 4 of the following genes; WASF3, CTNS, HIST1H2AG, ACOT7, LATPM4B, TGFB2, TPM1, H3F3A, APP, NGFRAP1, CLEC1B, HIST1H1A, SRP72, C20orf103 and CRYM.

7. The method of claim 5 wherein the biomarker subset comprises the following genes; HIST1H1A, SRP72, C20orf103 and CRYM.

8. The method of claim 1, wherein the step of measuring the expression values is achieved by measuring fluorescence intensities.

9. The method of claim 1, wherein the step of performing analysis to identify differentially expressed genes is achieved with use of a combination of the Kruskal-Wallis, non-parametric one-way analysis of variance, nonparametric Wilcoxon rank-sum test and Non-Parametric Linear Discriminant Analysis with a leave-one-out cross-validation analysis.

10. The method of claim 1, wherein the step of validating the identity of differentially expressed genes is achieved with use of Non-Parametric Linear Discriminant Analysis with a leave-one-out cross-validation analysis.

11. A method to distinguish thrombocytosis cohorts, the method including the following steps:
obtaining a hematologic sample from a subject;
determining gene expression of a biomarker subset;
analyzing gene expression of the biomarker subset; and
classifying the subject into a cohort.

12. The method of claim 11 wherein the gene expression of the biomarker subset comprises gene expression of a 4 biomarker subset.

13. The method of claim 11 wherein the gene expression of the biomarker subset comprises gene expression of a 11 biomarker subset.

14. The method of claim 11 wherein the gene expression of the biomarker subset comprises gene expression of a 15 biomarker subset.

15. The method of claim 14 wherein the biomarker subset comprises at least 4 of the following genes; WASF3, CTNS, HIST1H2AG, ACOT7, LATPM4B, TGFB2, TPM1, H3F3A, APP, NGFRAP1, CLEC1B, HIST1HIA, SRP72, C20orf103, and CRYM

16. The method of claim 15 wherein the biomarker subset comprises the following genes; HIST1HIA, SRP72, C20orf103, and CRYM

17. The method of claim 11 wherein the cohort is selected from the group consisting of: normal subjects, subjects with Essential Thrombocythemia (ET) and subjects with Reactive Thrombocytosis (RT).

18. The method of claim 11 wherein the step of determining gene expression is achieved through a microarray.

19. The method of claim 11 wherein the step of determining gene expression is achieved through a polymerase chain reaction (PCR).

20. The method of claim 19 wherein the PCR is a quantitative real-time reverse-transcription polymerase chain reaction (qRT-PCR).

21. The method of claim 11 wherein the step of determining gene expression is achieved through a microsphere based platform.

22. The method of claim 11 wherein the hematologic sample is whole blood.

23. The method of claim 11 wherein the hematologic sample is platelets.

24. The method of claim 11 wherein the subject is a human.

25. The method of claim 11 wherein the step of classifying the subject into a cohort is achieved by classifying a subject into the cohort with the highest posterior possibility.


SEQUENCE LISTING (30 sequences)

SEQ ID NO: 1 - DNA, 24 bp, Homo sapiens
aggatgggctgaagttctatactg

SEQ ID NO: 2 - DNA, 24 bp, Homo sapiens
attttccttctctcccactcttct

SEQ ID NO: 3 - DNA, 24 bp, Homo sapiens
catctggagtacaggacatagctc

SEQ ID NO: 4 - DNA, 24 bp, Homo sapiens
tgtgaggtggtctcaagtacaaat

SEQ ID NO: 5 - DNA, 24 bp, Homo sapiens
taaaatgtctgcctcacagatagg

SEQ ID NO: 6 - DNA, 24 bp, Homo sapiens
taaccctcctttacagaaaactgc

SEQ ID NO: 7 - DNA, 21 bp, Homo sapiens
gagaccgaggacgagaagaag

SEQ ID NO: 8 - DNA, 23 bp, Homo sapiens
ctctcaatgtgaattgggttttt

SEQ ID NO: 9 - DNA, 24 bp, Homo sapiens
cttgtttgttgctgaaatgctact

SEQ ID NO: 10 - DNA, 24 bp, Homo sapiens
aaagcagacttctaggtccatcag

SEQ ID NO: 11 - DNA, 24 bp, Homo sapiens
gtaaagtcttgcaaatgcagctaa

SEQ ID NO: 12 - DNA, 24 bp, Homo sapiens
acaaacagaacacaaacttccaaa

SEQ ID NO: 13 - DNA, 24 bp, Homo sapiens
ttaaacacctgcttaccccttaaa

SEQ ID NO: 14 - DNA, 24 bp, Homo sapiens
gcacactgtgttgctaaactctct

SEQ ID NO: 15 - DNA, 24 bp, Homo sapiens
agaatccactatgatgggaaacat

SEQ ID NO: 16 - DNA, 24 bp, Homo sapiens
aaattcacacacaaatgaaaatgg

SEQ ID NO: 17 - DNA, 24 bp, Homo sapiens
caacctacaagttctttgagcaga

SEQ ID NO: 18 - DNA, 24 bp, Homo sapiens
tcaagttcaggcatctacttgtgt

SEQ ID NO: 19 - DNA, 24 bp, Homo sapiens
ggaaatattcatggaggagatgag

SEQ ID NO: 20 - DNA, 24 bp, Homo sapiens
aaaatgcaaggaaaaagaaaacag

SEQ ID NO: 21 - DNA, 24 bp, Homo sapiens
aaaccaaataggaaactcaaatgc

SEQ ID NO: 22 - DNA, 24 bp, Homo sapiens
attaaacccatttcaaggtgagaa

SEQ ID NO: 23 - DNA, 24 bp, Homo sapiens
tctactgcagaaaagaagacgaga

SEQ ID NO: 24 - DNA, 24 bp, Homo sapiens
gcaaaagaggagatgggtaatgta

SEQ ID NO: 25 - DNA, 24 bp, Homo sapiens
tagggaaacatcttcctcaaagtc

SEQ ID NO: 26 - DNA, 24 bp, Homo sapiens
ctgtttaaatcctgggaaacctta

SEQ ID NO: 27 - DNA, 24 bp, Homo sapiens
cctatacagttgtcaatgcacaca

SEQ ID NO: 28 - DNA, 24 bp, Homo sapiens
acaggaagaagtccaaacaactct

SEQ ID NO: 29 - DNA, 24 bp, Homo sapiens
gaatattgctgctggttctcataa

SEQ ID NO: 30 - DNA, 24 bp, Homo sapiens
tcagggaaatataaagggaaatga